(19) United States
(12) Patent Application Publication
(10) Pub. No.: US 2004/0192947 A1
(43) Pub. Date: Sep. 30, 2004

(54) Mesostructured catalyst incorporating particles of nanometric dimensions

(76) Inventors: Jean-Yves Chane-Ching, Eaubonne (FR); Sylvaine Neveu, Paris (FR); Anne-Marie Le Govic, Paris (FR)

Correspondence Address: Jean-Louis Seugnet, Rhodia Inc, Intellectual Property Department, 259 Prospect Plains Road CN 7500, Cranbury, NJ, 08512-7500, US

(21) Appl. No.: 10/466,592

(22) PCT Filed: Jan. 17, 2002
(86) PCT No.: PCT/FR02/00190

(30) Foreign Application Priority Data
Jan. 18, 2001 (FR) 01/00698

(51) Int. Cl. C07D 301/06; B01J 23/00

(52) U.S. Cl. 549/533; 502/302

(57) ABSTRACT

The invention concerns a heat-stable mesostructured material, for use as heterogeneous catalyst, wherein the mesostructured walls comprise: (a) a mineral matrix; and (b) dispersed within said mineral matrix (a), particles of nanometric dimensions based on at least a rare earth T and on at least a transition element M different from said rare earth. The invention also concerns a method for obtaining such a material.

The present invention relates to thermally stable, mesostructured or ordered mesoporous materials that can be used especially in heterogeneous catalysis.

In the strict sense of the term, mesoporous materials are solids having, within their structure, pores having an intermediate size between that of micropores of zeolite-type materials and that of macroscopic pores.

More precisely, the expression “mesoporous material” denotes a material that has specifically pores of a mean diameter between 2 and 50 nm, denoted by the term “mesopores”. Typically, these compounds are compounds of the amorphous or paracrystalline silica type in which the pores are generally randomly distributed with a very broad pore size distribution.

As regards the description of such materials, reference may especially be made to Science, Vol. 220, pp. 365-371 (1983) or to Journal of the Chemical Society, Faraday Transactions, 1, Vol. 81, pp. 545-548 (1985).

As regards “structured” materials, these are materials that have an organized structure and are characterized more precisely by the fact that they have at least one scattering peak in an X-ray or neutron radiation scattering plot. Such scattering plots and the method of obtaining them are for example described in Small-Angle X-ray Scattering (Glatter and Kratky, Academic Press, London, 1982).

The scattering peak observed in this type of plot may be associated with a repeat length characteristic of the material in question, which will be denoted in the rest of the present description by the term “spatial repeat period” of the structured system.

On the basis of these definitions, the term “mesostructured material” is understood to mean, within the context of the invention, a structured porous material having a spatial repeat period of between 2 and 50 nm.

As a particular example of mesostructured materials, mention may be made of ordered mesoporous materials. These are mesoporous materials that have an organized spatial arrangement of the mesopores present in their structure and consequently actually have a spatial repeat period associated with the appearance of a peak in a scattering plot.

The family of materials of the generic name “M41S”, described in particular by Kresge et al. in Nature, Vol. 359, pp. 710-712 (1992) or by Q. Huo et al. in Nature, Vol. 368, pp. 317-321 (1994), constitutes the most well known example of mesostructured or ordered mesoporous materials: they are silicas or aluminosilicates whose structure is formed from ordered two-dimensional or three-dimensional channels in a hexagonal arrangement (MCM-41) or cubic arrangement (MCM-48), or materials that possess a vesicular or lamellar structure (MCM-50).

It should be noted that, although they are formed from a structure having channels and not mesopores, the compounds MCM-41 and MCM-48 are generally described in the literature as being ordered mesoporous materials. For example, Fengxi Chen et al. in fact describe, in Chemical Materials, Vol. 9, No. 12, p. 2685 (1997), the channels present within these structures as “two-dimensional or three-dimensional mesopores”.

On the other hand, the materials of vesicular or lamellar structure of the MCM-50 type cannot be likened to mesoporous structures, insofar as their porous parts cannot be regarded as mesopores. They are therefore denoted only by the term “mesostructured materials” in the rest of the description.

The mesostructured and ordered mesoporous materials, and in particular the materials of the M41S type defined above, are generally obtained by a process called LCT (liquid crystal templating). The LCT templating process consists in forming a mineral matrix such as a silica gel from mineral precursors in the presence of surfactant-type amphiphilic compounds, generally denoted by the term “templating” agents.

The expression “liquid crystal templating” stems from the fact that schematically it may be considered that the liquid crystal structure adopted by the surfactant molecules in the presence of the mineral precursors impresses on the mineral matrix its final form.

In other words, it may be considered that, within the liquid crystal structure, the mineral precursors are localized on the hydrophilic parts of the amphiphilic compounds before they condense together, thereby in fine conferring on the mineral matrix obtained a spatial arrangement copied over from that of the liquid crystal. By removing the surfactant, especially by a heat treatment or solvent entrainment, a mesostructured or ordered mesoporous material is obtained that constitutes the imprint of the initial liquid crystal structure.

On account of their high specific surface area and their particular structure, mesostructured materials are in general of great interest, especially in the field of catalysis, the field of absorption chemistry or the field of membrane separation.

However, the stable mesostructured materials known at the present time essentially consist of silica, titania, zirconia and/or alumina, and the attempts made to obtain mesoporous materials based on different constituents generally lead only to compounds of low stability, which precludes their use on an industrial scale.

The incorporation of chemical compounds capable of inducing specific properties, and especially beneficial properties in terms of catalysis, into an ordered mesoporous structure cannot be achieved in many cases.

The object of the present invention is to provide mesostructured materials that have, within their mesostructure, chemical compounds possessing intrinsic catalytic properties capable of inducing enhanced catalytic properties in the material, but without thereby affecting the stability of the mesostructure produced.

More precisely, the subject of the present invention is a thermally stable mesostructured material, the walls of the mesostructure of which comprise:

(a) a mineral matrix; and

(b) dispersed within this mineral matrix (a), nanoscale particles based on at least one rare earth E and on at least one transition element M different from this rare earth, in which particles:

(b1) the rare earth E is at least partly in the form of an oxide, hydroxide and/or oxyhydroxide, the transition element M then being at least partly in the oxidation state 0; or

(b2) the rare earth E and the transition element M are at least partly present in the form of a hybrid oxide possessing a crystal structure.

Advantageously, the mesostructured material of the present invention is a solid having at least one organized structure chosen from:

mesoporous mesostructures of P63/mmc three-dimensional hexagonal symmetry, P6 mm two-dimensional hexagonal symmetry or Ia3d, Im3m or Pn3m three-dimensional cubic symmetry; or

mesostructures of the vesicular, lamellar or vernicular type.

As regards the definition of these various symmetries and structures, reference may be made for example to Chemical Materials, Vol. 9, No. 12, pp. 2685-2686 (1997) or to Nature, Vol. 398, pp. 223-226 (1999).

In the most general case, the spatial repeat periods characterizing the mesostructure of the materials of the invention, determined by X-ray scattering methods (such as the method of small-angle X-ray scattering) or neutron scattering, are generally between 5 nm and 50 nm. Preferably, they are less than 20 nm and advantageously less than 15 nm. Advantageously, they are greater than 6 nm.

As regards the overall wall thickness of the mesostructures of the materials of the invention, this is generally between 2 nm and 20 nm. Advantageously, the wall thickness is greater than 3 nm and even more preferably greater than 4 nm. Thus, it may especially be between 3 and 15 nm, and advantageously between 4 and 10 nm.

In the particular case of ordered mesoporous structures, the mean pore diameter is generally between 2 and 30 nm. Advantageously, this mean diameter is less than 20 nm and more preferably less than 10 nm.

On account of their mesostructured character, the materials of the invention have, in the general case, a high specific surface area, generally between 500 and 3000 m²/cm³. Preferably, the materials of the invention have a specific surface area of greater than 600 m²/cm³ and advantageously greater than 800 m²/cm³.

The specific surface areas indicated above, expressed in units of area per unit of material volume, are calculated by multiplying the value of the BET specific surface area determined experimentally (using the Brunauer-Emmet-Teller method, described for example in The Journal of the American Chemical Society, Vol. 60, page 309 (February 1938) and corresponding to the NF T45007 standard), measured in m²/g, by the theoretical density of the material (expressed in g/cm³).

Apart from their mesostructured character, the materials of the invention are also characterized by their thermal stability.

Within the meaning of the invention, a mesostructured material is considered as thermally stable if its mesostructure is maintained up to a temperature of at least 400° C.

In this regard, it should be noted that, in the general case, the mesostructured materials of the prior art rarely exhibit such thermal stability. This is because exposing a mesostructured material to high temperatures generally leads to substantial embrittlement of the walls of the mesostructure, especially because of the crystallization of the mineral matrix, which is liable to reduce the wall thickness and induce stresses, which may result in the phenomenon of mesostructure collapse.

The present invention makes it possible, surprisingly, to provide very temperature-stable compounds.

Thus, the thermal stability of the materials of the invention is advantageously such that the mesostructure is maintained, even after a heat treatment at a temperature of greater than or equal to 500° C., or even greater than 600° C. In certain cases, it may even be possible to subject the materials of the invention to temperatures that may be up to 700° C., and even sometimes up to 800° C., without impairing the stability of their mesostructure.

As a general rule, especially so as to give the material substantial thermal stability, it is preferable for the mineral matrix (a) of the mesostructured material of the present invention to be based on silica, zirconia, alumina and/or titania.

Advantageously, the mineral matrix (a) is formed predominantly from silica. Thus, the mineral matrix (a) may advantageously be formed from silica, or from silica-alumina, silica-titanium and/or silica-zirconia blends, such blends then being characterized by silicon contents of greater than 50 mol %, preferably greater than 75 mol % and advantageously greater than 85 mol %.

Whatever its nature, it should be emphasized that, in the material of the invention, the mineral matrix (a) specifically acts as a binder between the particles (b). In other words, the particles (b) are present within the walls of the mesostructure, where they are dispersed in the binding matrix. In particular, it should therefore be emphasized that the materials according to the invention are especially to be distinguished from mesoporous materials that include particles in the internal space of their pores.

The particles (b) present within the walls of the materials of the invention are, specifically, nanoscale particles. Within the meaning of the invention, this term is understood to mean, in the most general sense, particles whose dimensions are between 1 and 200 nm.

Preferably, the particles (b) are particles of spherical or slightly anisotropic morphology, at least 50% of the population of which possesses a mean diameter of between 2 nm and 25 nm, advantageously between 3 and 15 nm, preferably with a monodisperse size distribution of these particles. Advantageously, these particles have, for at least 50% of their population, a mean diameter of less than 12 nm, and preferably less than 10 nm. Advantageously, this mean diameter may thus, for example, be between 3 and 10 nm in the case of at least 50% of the particles.

More particularly, the particles (b) may also be, in certain cases, in the form of highly anisotropic particles, of the rod type, provided that, for at least 50% of the population of these particles, the mean transverse diameter is between 1 and 25 nm, preferably between 2 and 20 nm, and provided that, for at least 50% of the particles, the length does not exceed 200 nm and advantageously remains less than 100 nm. Preferably, the particle size distribution is monodisperse.

It is also possible to envision, in certain cases, the use of particles (b) in the form of platelets, with a mean thickness of between 1 and 20 nm, preferably between 2 and 10 nm, and with mean large dimensions of between 20 and 200 nm, preferably less than 100 nm.

Advantageously, but in no way limitingly, the nanoscale particles present in the materials of the present invention are crystalline or partially crystallized particles, that is to say they have a degree of crystallinity, measured by X-ray diffraction, ranging from 2 to 100% by volume, this degree of crystallinity preferably being greater than 50% and advantageously greater than 10% or even greater than 20%. The presence of such partially crystallized particles within the mineral phase makes it possible to give the mesostructured materials of the invention, in addition to an ordered arrangement of their pore lattice and their thermal stability, an overall degree of crystallinity of their walls which is then generally greater than 5% by volume. Advantageously, the degree of crystallinity of the walls is then greater than 10% by volume, preferably greater than 20% by volume and particularly advantageously greater than 30% by volume.

The expression “overall degree of crystallinity”, as employed in the present description, denotes the degree of crystallinity of the walls of the structure, which takes into account in general both the possible crystallinity of the binding mineral phase and the crystallinity of the nanoscale particles included in this binding phase. It should therefore be noted that the notion of crystallinity of the material within the meaning of the invention relates specifically to the actual crystallinity of the walls of the material. Consequently, it should in particular be distinguished from the order presented, at a more macroscopic level, by the pore lattice of the mesoporous structure.

Specifically, the particles (b) incorporated into the binding mineral phase of the materials of the invention are particles based on a rare earth E and a transition element M different from the rare earth E.

The term “rare earth” denotes, within the meaning of the present invention, a metallic element chosen from yttrium and the lanthanides, the lanthanides being metallic elements whose atomic number is between 57 (lanthanum) and 71 (lutecium) inclusive.

As regards the term “transition element” this denotes, within the meaning of the invention, a metallic element chosen from columns Ib, IIb, IIIa (including the lanthanides), IVa, Va, VIa, VIIa and VIII of the Periodic Table published in the Supplement to the Bulletin of the French Chemical Society No. 1 (January 1966). In other words, it is an element whose atomic number is between 21 and 30, between 39 and 48 or between 57 and 80 inclusive.

According to a first advantageous variant, the particles (b) present within the mesostructured materials of the invention comprise:

an oxide, hydroxide and/or oxyhydroxide of the rare earth E; and

the transition element M, different from the rare earth E, at least partly in the oxidation state 0.

The expression “oxide, hydroxide and oxyhydroxide of the rare earth E” is understood to mean, within the context of the invention, an oxide, hydroxide or oxyhydroxide essentially incorporating cations of the rare earth E, and in which some of the cations of the rare earth E may optionally be substituted with at least one type of metal cation other than a cation of the rare earth E, especially with alkali metal cations (especially Li⁺, Na⁺ or K⁺), alkaline-earth metal cations (in particular Mg²⁺ or Ca²⁺), cations of transition elements as defined above (such as, for example, Ag⁺, Mra²⁺, Mn³⁺, Ce⁴⁺, Ce³⁺, Ti⁴⁺, Zr⁴⁺ cations) or else with aluminum or tin cations.

In this first variant, especially so that the combined presence of the compound of the rare earth E and of the transition element M gives the material beneficial catalytic properties, it is generally preferred for at least some of the transition element M present in the oxidation state 0 to be localized on the periphery of the particles (b). However, in this case it is preferred for the transition element M not to completely cover the particles (b). This is because it is likewise preferable for at least some of the rare earth oxide, hydroxide and/or oxyhydroxide to be present on the periphery of the particles (b).

Advantageously, according to this first variant, the rare earth E present in the cationic state in the oxide, hydroxide and/or oxyhydroxide, is chosen from yttrium, lanthanum, cerium, praseodymium, neodymium, samarium, europium, gadolinium, terbium or dysprosium and preferably from yttrium, lanthanum, cerium and europium. Moreover, the rare earth E is preferably at least partly present in the form of a rare earth oxide.

According to this first variant of the invention, the transition element M itself is preferably chosen from metals possessing, under standard temperature and pressure conditions, oxidation-reduction (corresponding metal/metallic cation(s)) potentials of greater than −0.3 V relative to a standard hydrogen electrode. Thus, the transition element M is advantageously chosen, in the first variant, from noble metals, such as rhodium, platinum, palladium and ruthenium, or else from other metals such as cobalt, copper, silver or nickel. However, the second variant of the invention is not limited to these particular metals and, in particular, it may be envisioned using metals possessing lower oxidation-reduction potentials, such as manganese for example. The transition element M may be present by itself in the oxidation state 0 or in combination with at least one other transition element in the 0 state.

Thus, as particularly advantageous examples of particles (b) that can be used according to the first variant of the invention, mention may especially be made of particles based on rare earth oxides that are “doped” by the presence of one or more metals in the oxidation state 0 and are denoted as (metal: rare earth oxide). Thus, mention may in particular be made of particles based on doped oxides of the type (Ni:CeO₂); (Ru:CeO₂); (Ni+Ru:CeO₂); (Pd:CeO₂); (Pd+Pt:CeO₂); (Mn:CeO₂); (Co:La₂O₃); (Mn:La₂O₃); (Pd:La₂O₃) (where lanthanum is partly substituted with cerium cations); (Pd:CeₓMn_{y}O_{z}) (where x is between 0.5 and 0.9; y is between 0.1 and 0.5; and z is between 1.8 and 3.2); (Co:Y₂O₃); or (Mn:Y₂O₃).

Whatever their precise composition, the particles (b) present in the particles (b) of the first variant of the invention are preferably characterized by a (metal M/rare earth E) molar ratio of between 0.002 and 0.2, and preferably between 0.005 and 0.1.

In the particular case when at least one other transition element other than the transition element M in the oxidation state 0 other than the metal M is present, the (metals in the oxidation state 0 [including the metal M]/rare earth E) molar ratio is advantageously between 0.002 and 0.2.

According to a second variant of the invention, the particles (b) present within the mineral matrix (a) are based on a mixed oxide possessing a crystal structure that incorporates cations of the rare earth E and cations of the said transition element M different from the rare earth E.

In the present description, the term “mixed oxide” denotes, in the broad sense, any oxide based on at least two different metallic elements. In particular, the mixed oxides specifically present in the particles (b) according to the second variant of the invention are mixed oxides based on cations of the rare earth E and of the transition element M, but they may incorporate other metal cations, and especially rare earth cations, transition metal cations, alkali or alkaline-earth metal cations, or else aluminum or tin cations.

Specifically, the mixed oxides used according to the second variant of the invention are oxides having a crystal structure. Thus, they may advantageously be oxides having a structure of the perovskite or pyrochlore type, or else a structure similar to that of K₂NiF₄. Particularly advantageously, they may have a perovskite structure.

According to the second variant of the invention, whatever the exact structure of the mixed oxides used, the rare earth E is advantageously chosen from yttrium, lanthanum, cerium, praseodymium, neodymium, samarium, europium, gadolinium, terbium and dysprosium, and preferably from lanthanum, cerium and yttrium. As regards the transition element M, this is preferably chosen from titanium, chromium, manganese, iron, cobalt, nickel, copper, zinc, zirconium and silver, or else a rare earth different from the rare earth E. Advantageously, the transition element M is chosen from manganese, iron, cobalt, nickel, copper and silver, or a rare earth different from the rare earth E.

Thus, as nonlimiting examples of mixed oxides that can be used according to the second variant of the invention, mention may be made of:

perovskites, of general formula ABO₃ (where A and B represent the two crystal sites characteristic of a perovskite structure, and in which cations of the rare earth E occupy sites of type A and in which cations of the metal M occupy sites of type B.

Advantageously, sites A and B of the perovskites of the invention may be partly occupied by cations of metals other than E and M, these being called “substituting cations”. However, in this case, the rare earth E advantageously remains predominant in the A sites (the percentage occupancy of the A sites by E thus preferably remains greater than 50%, advantageously greater than 60% and particularly preferably greater than 70%) and the metal M also preferably remains predominant within the B sites (the percentage occupancy of the B sites by M preferably remains greater than 50%, advantageously greater than 60% and particularly preferably greater than 70%). In the specific case in which the A or B sites are partially substituted, the substituting cations are advantageously at a degree of oxidation other than the cations of E or of M occupying the rest of these sites, which in particular leads to the creation of cationic-type or anionic-type vacancies, often useful in catalysis.

As examples of useful perovskites according to the invention, mention may especially be made of perovskites of formula TMO₃ (based only on the rare earth E and the metal M), and in particular LaMnO₃, LaCoO₃, LaCeO₃, LaFeO₃ or LaNiO₃, or else perovskites substituted on the A or B sites (sometimes called “doped” perovskites), such as La0.8Ag_{0.2}MnO₃, LaMn_{0.6}Cu_{0.4}O₃, LaCe_{0.75}Co_{0.25}O₃, La_{0.8}Mg_{0.2}FeO₃, or else LaNi₁₋ₓFeₓO₃, where x is between 0.02 and 0.4 inclusive;

pyrochlores of general formula A₂B₂O₇ (where A and B represent the two crystal sites characteristic of a pyrochlore structure) and in which cations of the rare earth E occupy sites of type A and in which cations of the metal M occupy sites of type B. Here again, these sites A and B may advantageously be partly occupied by substituting cations of the type of those described in the case of perovskites, their oxidation state being again advantageously chosen so as to create cationic or anionic vacancies within the pyrochlore crystal lattice. As in the case of perovskites, the rare earth E then advantageously remains predominant in the A sites and the metal M also preferably remains predominant within the B sites, with degrees of occupancy of the A sites by E generally of greater than 50%, advantageously greater than 60% and preferably greater than 70%; and with a degree of occupancy of the B sites by M of preferably greater than 50%, advantageously greater than 60% and even more preferably greater than 70%. As examples of useful pyrochlores according to the invention, mention may especially be made of compounds of formula E₂M₂O₇ (based only on a rare earth E and a metal M), and in particular Ce₂Sn₂O₇, and substituted pyrochlores, such as Ce_{1.2}Ca_{0.8}Sn₂O₇ or pyrochlores of formula Ce₂₋ₓSrₓSn₂O₇, in which x is between 0.2 and 1.2 inclusive;

mixed oxides of general formula A₂BO₄ having a structure similar to that of K₂NiF₄ (where A and B represent the two crystal sites characteristic of said structure) and in which cations of the rare earth E occupy sites of type A and in which cations of the metal M occupy sites of type B. Here again, the A and B sites may advantageously be partly occupied by substituting cations of the same type as those described in the case of perovskites, their oxidation state again being chosen advantageously so as to create cationic-type or anionic-type vacancies within the crystal lattice. As in the case of the perovskites and pyrochlores, the rare earth E then advantageously remains predominant in the A sites and the metal M also preferably remains predominant in the B sites, with degrees of occupancy of the A sites by E of generally greater than 50%, advantageously greater than 60% and preferably greater than 70%; and with a degree of occupancy of the B sites by M of preferably greater than 50%, advantageously greater than 60% and even more preferably greater than 70%. As examples of such mixed oxides, mention may especially be made of mixed oxides of the La₂NiO₄ type, where the A or B sites are optionally doped with silver, calcium strontium, magnesium, manganese, iron, cobalt, nickel, zirconium, molybdenum, cerium, gadolinium, praseodynium or europium cations.

In the second variant of the invention, whatever the nature of the mixed oxide, the particles (b) based on this mixed oxide may furthermore incorporate at least transition element M′ present in the oxidation state 0.

It is therefore generally preferable for at least part of the transition element M′ present in the oxidation state 0 to be localized on the periphery of the particles (b). Moreover, it is preferable for the transition element M′ not to completely cover the particles (b). This is because it is also generally preferable for at least part of the mixed oxide to be present on the periphery of the particles (b).

Particularly advantageously, the transition element M′ optionally present is chosen, as the case may be, from metals possessing, under standard temperature and pressure conditions, oxidation-reduction (metal/corresponding metal cation) potentials of greater than −0.3 V relative to a standard hydrogen electrode. Thus, the transition element M′ is advantageously chosen from rhodium, platinum, palladium or ruthenium, silver, cobalt, copper or nickel. However, it is possible in particular to envision the use of other metals possessing lower oxidation-reduction potentials, such as manganese for example.

Whatever the nature of the transition element M′, the (metal M′/(rare earth E+metal M)) molar ratio is, when such an element in the oxidation state is present, advantageously between 0.002 and 0.05.

Whatever the exact nature of the particles (b) incorporated into the matrix (a), it should be noted that the mesoporous materials incorporating them are capable of exhibiting useful properties in catalysis. Especially so as to optimize these catalytic properties, it is therefore often preferable, in the most general case, for at least some of the particles incorporated into the binding mineral phase to be in contact with the porous parts forming the internal space (pores, channels, etc.) of the mesostructured material, especially so as to optimize the benefit of the properties induced by the presence of the particles (b). In other words, the material according to the invention is preferably a material in which the mineral phase (a) actually acts as an inter-particle binder, that does not completely coat the particles (b) that it binds together.

Moreover, if the presence of the particles (b) is important in order to give the material of the invention its specific properties, it is also important to consider the paramount role of inter-particle consolidation played by the mineral phase. In this regard, it should be noted that it is often preferable, so as to ensure sufficient stability of the material, for the (particles)/(particles+mineral matrix) volume ratio to be less than 95%.

In the materials of the invention, this (particles) (particles+mineral matrix) volume ratio is generally between 5 and 95%. Preferably, this ratio is at least equal to 10% by volume, particularly advantageously at least equal to 20% by volume and even more preferably at least equal to 30% by volume. Moreover, it is preferably less than 90% by volume, and advantageously less than 85% by volume. Thus, this ratio may advantageously be between 40% and 80% by volume.

According to a second aspect, the subject of the present invention is also a process for preparing the mesostructured materials described above. This process is characterized in that it comprises the steps consisting in:

(1) bringing together, in a medium containing a templating agent:

(i) nanoscale particles comprising at least one oxide, hydroxide or oxyhydroxide of the rare earth E, said particles being complexed by at least one salt of the metal M; or nanoscale particles comprising at least one mixed oxide or mixed hydroxide of the rare earth E and of the metal M; and

(ii) a mineral precursor, capable of leading, in said medium, to the formation of an insoluble mineral phase under suitable pH conditions;

(2) leaving said mineral phase to form from said precursor (ii), or if necessary to make said mineral phase form by adjusting the pH, whereby a mesostructure whose walls are formed from said mineral phase trapping at least some of the initially introduced nanoscale particle is obtained;

(3) if necessary, subjecting the mesostructure obtained from step (2) to a heat treatment step and/or to a reduction step, this or these steps being carried out, as the case may be, so that, after this or these treatments, the particles present within the walls of the mesostructure comprise particles meeting the definition of the particles (b) that was given above in the case of the materials of the invention; and

(4) if necessary, removing the templating agent optionally present in the pores of the mesostructure obtained from these various steps.

At the end of the process, the product obtained is recovered.

The medium used in the process of the invention is preferably an aqueous medium, but it may also, in certain cases, be an aqueous alcoholic medium, and preferably in this case a water/ethanol medium. When a water/alcohol medium is used, the ratio of the volume of alcohol to the volume of water, these being measured before mixing, is generally less than or equal to 1.

The term “templating agent” is understood to mean, within the present invention, a surfactant-type amphiphilic compound (or a blend of such amphiphilic compounds) used in an amount such that it forms micelles or a liquid crystal phase organized within the medium in which the templating procedure of the process of the invention takes place. The concentrations of templating agent to be used in the medium in order to obtain formation of micelles or of an organized liquid crystal phase depend on the exact nature of the templating agent. It lies within the competence of those skilled in the art to adapt this concentration according to the phase diagram of the templating agent(s) adopted. Whatever its nature, the templating agent leads to an LCT templating procedure as defined above. In other words, the particles (i) and the mineral precursor (ii) are localized within the hydrophilic regions of the templating agents organized in micelles or in a liquid crystal phase, which, upon formation of the mineral phase, leads to a mesostructure being obtained, the walls of which are formed by particles bonded together by the binding mineral phase formed.

In general, most amphiphilic compounds may be used as templating agents in the process of the invention. However, it is generally preferable to use nonionic templating agents. As indicative and nonlimiting examples of nonionic templating agents that can be used in the process of the invention, mention may be made of nonionic amphiphilic agents of the block copolymer type, and more particularly PEO/PPO/PEO (poly(ethylene oxide)/poly(propylene oxide)/poly-(ethylene oxide)) triblock copolymers of the type of those described especially by Zhao et al. in Journal of the American Chemical Society, Vol. 120, pp. 6024-6036 (1998) and sold under the generic brand name of Pluronic® by BASF. Advantageously, nonionic surfactants may also be used, such as grafted-poly(ethylene oxide)-based surfactants, of the type of poly(ethylene glycol) ethers sold under the brand names Brij®, Tergitol® or Triton® by Aldrich or Fluka, or else of the type of nonionic surfactants having a sorbitan head, of the type of those sold by Fluka under the brand names Span® or Tween®.

When the above polyethoxylated templating agents are used, the amounts of amphiphilic agents employed are generally such that, in the medium where the templating procedure takes place, the ratio of the molar concentration of (CH₂CH₂O) units present in the amphiphilic agents to the sum of the molar concentrations of metal cations present in the particles (i) and in the mineral precursor (ii) is between 0.05 and 3, and preferably between 0.1 and 2.

In the most general case, whatever the exact nature of the templating agents employed, the (templating agent)/(metal cations present in the particles (i)+mineral precursor (ii)) molar ratio is generally between 0.05 and 3. In the general case, this ratio is preferably less than 2.

Moreover, it should be noted that, especially so as to improve the homodispersity of the liquid crystal phase formed during the templating procedure, and so as in fine to achieve effective incorporation of the particles (i) within the binding phase, it is often preferable to use a blend of several types of templating agent, and especially, where appropriate, ionic surfactant/nonionic surfactant blends.

The particles (i) are generally introduced in step (1) of the process in the form of colloidal dispersions within which the particles are preferably not agglomerated and advantageously have a monodisperse particle size distribution. Advantageously, the colloidal particles present in these dispersions have, for at least 50% of their population, a mean diameter, determined for example by analyzing micrographs obtained in transmission electron microscopy, of between 1 nm and 25 nm and preferably between 2 and 15 nm. This maximum mean diameter presented by at least 50% of the population is advantageously less than 10 nm, and advantageously greater than 3 m. Thus, it may advantageously be between 3 and 8 nm. Advantageously, the particles (i) used have a degree of crystallinity, measured by X-ray diffraction, of between 2 and 100% by volume. Preferably, this degree of crystallinity is greater than 5% by volume.

Preferably, the concentration of particles in the suspensions used according to the invention, that is to say the amount of constituent compound of said particles contained in one liter of said suspensions, is generally greater than 0.25 mol per liter and is advantageously greater than 0.5 mol per liter. Generally, this concentration is less than 4 mol per liter.

The particles (i) based on an oxide, hydroxide or oxyhydroxide of the rare earth E and complexed with at least one salt of the metal M, that can be used according to a first way of implementing the process of the invention, may be obtained (A) obtained by various methods. For example, it is possible to form an aqueous blend comprising a lanthanide salt and a complexing agent. The complexing agents used then generally have a dissociation constant for a complex with the lanthanide cation of greater than 2.5. In a second step, the blend is then basified by adding a base of the aqueous ammonia type until obtaining a pH whose value is to be adapted according to the nature of the lanthanide and to the amount of complexing agent. In particular, it should be noted that the pH is lower the higher the content of complexing agent. This operation is generally carried out until a pH is obtained at which the dissolution of the precipitate formed in the first step of the basification step starts to be observed. A salt of metal M, or a metal complex (for example Pd(NH₃)₂ (NO₃)₂ or Pd(NO₃)) is then generally added in an appropriate amount to the lanthanide dispersion obtained beforehand. The pH is readjusted within the pH range close to the pH of the lanthanide dispersion. The final step of the process then generally consists of a heat treatment, also called thermohydrolysis, that consists in heating the mixture obtained from the previous step. After this treatment, a colloidal lanthanide/metal M oxyhydroxide dispersion is obtained.

The particles (i) based on a mixed oxide or mixed hydroxide of the rare earth E and of the metal M, that can be used according to a second method of implementation, may be obtained especially by a method consisting, for example, in forming an aqueous mixture comprising a lanthanide salt, a salt of metal M and a complexing agent. The complexing agents generally used in this case are those having dissociation constants for complexes with the lanthanide cation and the cation of metal M of greater than 2.5. In a second step, the mixture is basified using a base such as aqueous ammonia, until obtaining a pH whose value varies depending on the nature of the lanthanide, on the cation of metal M and on the content of complexing agent. Here again, it should be noted that the pH is lower the higher the content of complexing agent. Generally the operation is carried out until a pH is obtained at which dissolution of the precipitate formed in the first part of the basification step starts to be observed. The final step of the process is then generally, once again, a thermohydrolysis, consisting in heating the basified mixture obtained from the previous step. After this treatment, a colloidal mixed lanthanide/element M oxyhydroxide dispersion is obtained.

In the most general case, the process of the invention is advantageously carried out either by mixing the particles (i) in the form of a stable colloidal dispersion with a solution containing the metal precursor (ii) and the templating agent, or by mixing a solution containing the metal precursor (ii) with a stable colloidal dispersion containing the particles (i) and the templating agent.

According to an advantageous variant, the pH of the colloidal dispersion and the pH of the solution containing the precursor are chosen so that when they are mixed a medium is obtained whose pH causes the mineral phase to be precipitated from the precursor.

According to another variant, the mixture produced may also lead to the formation of a medium whose pH is such that the precursor remains stable. An acid (or a base) is then added to the medium so as to lower (or alternatively increase) the pH until reaching a value for which the mineral matrix forms.

Whatever the variant employed, it is preferable, especially so as to obtain optimum binding of the particles by the mineral matrix being formed, for the colloidal particles used not to be able to agglomerate at the various pH values at which they are used. Thus, it is preferable for colloidal stability to be ensured throughout the length of the process, and most particularly in the colloidal dispersion initially introduced, and in the medium within which the precipitation of the mineral phase takes place. Depending on the medium employed, the surface of the nanoscale particles used may, for example, consequently be optionally modified, especially so as to stabilize the dispersion and prevent or limit the phenomena of floculation during the templating procedure. Thus, when the process has to be carried out in basic medium, the surface of the particles may then, for example, be modified by the presence of anions of organic acids such as, for example, citrate, acetate or formate anions, giving the particles negative surface charges.

Whether or not this condition relating to the stability of the particles at the various pH values employed is satisfied, it is often preferable, especially so as to achieve effective incorporation of the particles within the mineral phase, for the mineral phase to form as rapidly as possible. For this purpose, the mineral phase is therefore generally formed from the precursor (ii) by a sudden change in the pH of the medium, from a pH value at which the mineral precursor is soluble to a pH value at which the mineral material precipitates. However, the process of the invention is not limited to the use of this particular method of implementation.

The term “mineral precursor” is understood within the context of the invention to mean a mineral or organometallic compound capable of leading, under suitable pH conditions, to the formation of a mineral matrix such as silica.

The exact nature of the mineral precursor (ii) introduced in step (1) of the process of the invention therefore depends, of course, on the mineral phase that it is desired to form in order to ensure inter-particle bonding within the final material. However, insofar as the binding mineral phase formed following the addition of the mineral precursor of the step generally consists at least predominantly of silica, the mineral precursor used is advantageously a silicate, preferably an alkali metal silicate and advantageously a sodium silicate. This silicate may then be used with other mineral precursors, such as a titanium oxychloride or a sodium aluminate or a zirconium oxychloride or a zirconium oxynitrate, whereby in fine binding mineral phases of the SiO₂—TiO₂, SiO₂—Al₂O₃ or SiO₂-ZrO₂ type are obtained.

Within the specific context of a silicate precursor, silica polycondensation is observed when the silicate is exposed to a pH of between 5 and 10. However, especially so as to increase the rate of polycondensation of the silica, it is advantageously preferable for the silica matrix to form from the silicate at a pH of between 5.8 and 9.5, preferably at a pH of less than 8.5, and advantageously at a pH of between 6 and 8.5.

Thus, within the specific context of using a silicate as precursor (ii), the templating procedure may, according to a first method of implementation, be carried out by mixing, generally performed instantly, a colloidal dispersion of particles (i) with an acid aqueous medium of pH between 1 and 3.5 containing the silicate essentially in the silicic acid state. The acid medium containing silicic acid used in this case advantageously has a pH of between 1.5 and 2.5 and is generally obtained by adding, preferably instantly, a suitable amount of a solution of a strong acid, especially hydrochloric acid or nitric acid, to a solution of an alkali metal silicate having an initial pH of generally greater than 10, at which the silicate is stable and does not lead to silica polycondensation. The templating agent is generally incorporated into the acid medium thus obtained before mixing with the colloidal dispersion, but the templating agent may also, according to a variant, be incorporated beforehand into the colloidal dispersion, which will then be mixed with the acid medium.

According to this first method of implementation, whether the templating agent is initially present within the colloidal dispersion or at the in the medium containing the mineral precursor, it is generally preferable to adjust the pH of the colloidal dispersion to a pH such that the mixing of the dispersion with the silicic acid solution leads directly to a medium of pH between 5.8 and 9.5 being obtained. For this purpose, the pH of the colloidal dispersion is between 6 and 10.5, and preferably between 6 and 10. According to this first method of implementation, it is often advantageous, after the colloidal dispersion has been mixed with the acid medium containing the templating agent, to add a salt such as, for example, NaCl, NH₄Cl or NaNO₃, especially to increase the rate of silica formation.

According to a second possible method of implementation when the process of the invention is specifically carried out using a silicate precursor, it is also possible to add a colloidal dispersion having a pH of generally between 6 and 10.5 to a medium of pH greater than 10 containing the silicate in the stable state and the templating agent. As a general rule, a medium of pH greater than 9, generally between 9.5 and 12.5, containing the templating agent, the stable silicate and the particles (i) is then obtained. Colloidal stability of the specific particles used in the process of the invention is generally ensured in this pH range. According to this second method of implementation, the templating procedure is generally initiated by adding an acid to the medium thus obtained, so as to reduce the pH to a value of between 6 and 9.5.

Another particular method of implementation that can be envisioned when it is desired to form a mineral matrix based on silica consists in using a silicon alkoxide of the tetraethyl orthosilicate type as mineral precursor (ii). In this case, silica formation preferably takes place in acid medium, advantageously at a pH of less than 3. In this case, a solution of a silicon alkoxide of the tetraethyl orthosilicate type in the stable state is therefore generally added to a colloidal dispersion of low pH, generally less than 3.

In the most general case, whatever the mineral precursor used and whatever the colloidal particles used, the templating procedure is advantageously carried out at room temperature or at a temperature at above room temperature, preferably at a temperature between 15° C. and 90° C., and particularly preferably between 20° C. and 65° C. It is within the competence of those skilled in the art to adapt this temperature parameter according to the nature of the templating agent used and the spatial arrangement of the ordered mesoporous material that it is desired to obtain, according to the phase diagram presented by the structuring agent employed.

In practice, step (2) of forming the mineral phase from the mineral precursor generally comprises a maturing step. The duration of this step may vary and depends, of course, on the nature of the mineral precursor (ii) used. It is within the competence of those skilled in the art to adapt this duration, which is typically around 1 to 20 hours depending on the precursor used. This maturing step is generally carried out at a temperature between 15° C. ad 85° C. and preferably at a temperature between 20° C. and 60° C.

In the general case, what is obtained after the templating procedure and this optional maturing step is a mesostructured solid, the walls of the mesostructure of which includes some of the initially introduced particles (i), these being bonded together by the mineral matrix formed from the precursor (ii), and in which solid the porous parts are occupied by molecules of the templating agent. The material obtained may then optionally be subjected to a washing step using a solvent, especially water or an organic solvent, and/or to a drying step.

To obtain a porous mesostructure material as defined above, it is sometimes necessary to subject the solid obtained after step (2) to a heat treatment and/or reduction step (3) so as to modify the structure or the chemical nature of the particles incorporated into the walls.

This is in particular the case in the specific context of the preparation of the materials described above when they incorporate, within their walls, particles (b) comprising both an oxide, hydroxide or oxyhydroxide of the rare earth E and the metal M in the oxidation state 0. In this case, it is general practice to use particles (i) comprising at least one oxide, hydroxide or oxyhydroxide of the rare earth E, said particles being complexed by at least one salt of the metal M, or else mixed oxides and/or hydroxides of the rare earth E and of the transition element M. So as to obtain, within the walls of the material, particles comprising both an oxide, hydroxide or oxyhydroxide of the rare earth E and the metal M in oxidation state 0, it is necessary, in this case, to subject the structure obtained after step (2) to a heat treatment and reduction step (3) carried out to as to form metal M in the oxidation state 0 from at least some of the salt of the metal M. Preferably, this heat treatment and reduction step comprises:

either a heat treatment carried out in an inert atmosphere (for example in argon or in nitrogen) and/or in an oxidizing atmosphere (and especially in air), followed by a second heat treatment in a reducing atmosphere (and especially in an Ar/H₂ mixture);

or a direct heat treatment in a reducing atmosphere (and especially in an Ar/H₂ mixture).

According to one particular method of implementation, specific particles (i) incorporating organic additives of the acetate, citrate or polyacrylate type may be employed. In this case, the heat treatment/reduction step is carried out by a heat treatment in an inert or oxidizing atmosphere of the argon, nitrogen or air type.

The exact conditions for carrying out the heat treatment/reduction step is, of course, to be adapted according to the exact nature of the particles employed and in particular the nature of the elements M and E used. It is within the competence of the those skilled in the art to adapt the parameters of the heat treatment/reduction step according to the oxidizing and reducing characteristics of the compounds present in the material, so as to obtain the desired particles within the material. In the general case, the various heat treatments (whether in an inert, oxidizing or reducing atmosphere) are advantageously carried out with a progressive temperature rise profile, preferably with a rate of temperature rise of between 0.2 and 5° C. per minute, and advantageously of less than 2° C. per minute, so as to avoid embrittling the material. These heat treatments are generally carried out up to temperatures of between 250° C. and 600° C., typically around 300 to 400° C., at which the material is generally maintained for between 1 and 8 hours, which generally also leads to the templating agent present within the mesostructure being eliminated.

Within the context of the preparation of a material comprising within its walls particles (b) comprising a mixed oxide of the rare earth E and of the metal M having a crystal structure of the aforementioned perovskite or pyrochlore type, the particles (i) used comprise at least one mixed oxide or mixed hydroxide of the rare earth E and of the metal M. If these particles (i) do not have a crystal structure, a heat treatment step (3) must specifically be carried out. As a general rule, this heat treatment is carried out in an inert or oxidizing atmosphere, and advantageously with a progressive temperature rise profile, preferably with a rate of temperature rise of between 0.1 and 5° C. per minute, advantageously less than 2° C. and preferably between 0.1 and 1° C. per minute. These heat treatments are generally carried out at temperatures lying typically lying between 250 and 400° C., at which the material is generally maintained for between 1 and 8 ours, thereby generally leading, here again, to the templating agent present within the mesostructure being eliminated. It should be noted that, even when it is unnecessary to modify the structure of the particles introduced, such a heat treatment step is often recommended, especially so as to consolidate the mesostructure obtained.

In the particular case of the preparation of the material incorporating within its walls particles comprising both a mixed oxide with a crystal structure based on the rare earth E and on the metal M, and a metal M′ in the oxidation state 0, the particles (i) employed generally comprise at least one mixed oxide or mixed hydroxide of the rare earth E and of the metal M, and these particles are complexed by at least one salt of the metal M′. In this case, a heat treatment/reduction step (3) is necessary so as to make at least some of the metal M′ introduced in salt form pass to the oxidation state 0. Moreover, this heat treatment step is then carried out so that, after the treatment, the particles present within the walls of the mesostructure comprise a mixed oxide of the rare earth E and of the metal M having a crystal structure. As a general rule, the heat treatment or treatments carried out in this particular case are of the type of the heat treatment steps described above, with a progressive temperature rise profile, preferably with a rate of temperature rise of between 0.1 and 5° C. per minute, generally carried out up to temperatures lying typically lying between 300 and 400° C., at which the material is generally maintained for between 1 and 8 hours.

Whether or not a heat treatment and/or reduction step (3) is carried out, the templating agent present in the mesostructure solid after the templating procedure must specifically be eliminated in order for a material useful in terms of catalysis to be obtained.

If a heat treatment and/or reduction step (3) is specifically carried out, the templating agent is generally eliminated de facto during the heat treatment step of step (3). This is because, as already emphasized, the heat treatment of step (3) generally induces, at the same time as the desired modification of the particles present, the thermal elimination of the templating agent.

If such a step is not carried out, or else when the operating conditions for this step are insufficient to eliminate the templating agent, a templating agent elimination step (4) may be necessary. This step (4) may especially be carried out, where appropriate, by a heat treatment. In this case, the heat treatment is advantageously carried out with a temperature rise profile of between 0.2° C. per minute and 5° C. per minute, and preferably with a temperature rise profile of between 0.5° C. per minute and 2° C. per minute, so as not to degrade the material. This temperature rise is carried out up to a temperature allowing elimination of the templating agent, that is to say generally up to a temperature of between 250° C. and 600° C., and typically at least equal to 350° C. The material is then generally maintained at this temperature for a time advantageously of between 1 and 8 hours. This heat treatment step may be carried out firstly in an inert atmosphere, especially in argon or nitrogen, then secondly in an oxidizing atmosphere, and especially in air. In certain cases, it may be carried out directly in an oxidizing atmosphere, and advantageously in air, where appropriate.

According to another variant, which may especially be carried out when a heat treatment and/or reduction step (3) is unnecessary, the templating agent may also be eliminated by solvent entrainment. It should be noted that solvent entrainment is facilitated by the fact that an amphiphilic compound of the nonionic surfactant type is preferably used, thereby inducing a templating agent/matrix interaction weak enough to allow this type of elimination.

Whatever the method of eliminating the templating agent adopted, the solid obtained may furthermore be subjected to a subsequent heat treatment, and especially to a calcining treatment. The object of this optional additional heat treatment is especially to locally consolidate the walls of the mesostructure and increase the crystallinity of the material.

In this regard, it should nevertheless be stressed that, although the heat treatment of a mesostructured material may induce local mechanical consolidation, it may also, on the down side, lead to a reduction in the wall thickness, which generally results in overall embrittlement of the mesostructure obtained.

Depending on the compositions of the binding mineral phase and of the nanoscale particles employed, it lies within the competence of those skilled in the art to carry out the heat treatment or not, and to adapt, where appropriate, the temperatures to which the material is subjected, so as not to impair its final stability.

In this regard, it should however be emphasized that, because of the particular structure of the materials of the invention, a heat treatment without excessive embrittlement is easier to achieve in the case of mesostructured materials of the present invention than in the case of the usual mesostructured materials described in the prior art.

This is because the particular process used in the present invention, together with the specific use of nanoscale particles in the production of the materials of the invention, results, after the templating agent elimination step, in mesostructured materials having much larger wall thicknesses than in the case of the mesostructures that are obtained conventionally. In fact, the process of the present invention makes it possible to obtain extremely stable mesostructured materials that have, even after heat treatment, wall thicknesses of typically between 3 and 10 nm.

Moreover, it should be noted that, during the optional heat treatment steps, a reaction may be observed between the chemical species present in the particles and the binding mineral phase that contains them. This reaction has the consequence of modifying the chemical nature of the mineral phase. Thus, in the case of a material incorporating the particles in a silica-type mineral phase, the heat treatment may result in some cases in the formation of a silicate of the rare earth E and of the metal M within the mineral phase.

In general, it remains to specify that, in some cases, it may happen that the nanoscale particles obtained are completely covered by the mineral matrix. If it is desired, in the final material, for the particles not to be completely coated with the matrix, the material obtained may furthermore undergo a partial chemical etching of the mineral phase, especially by alkaline compounds of the NH₄OH or NaOH type or else by hydrofluoric acid. In this case, it lies within the competence of those skilled in the art to adjust the concentration of hydroxyl or fluoride ions and the duration of the treatment and the temperature employed, so as to control the dissolution of the mineral phase. Under these conditions, the post-treatment allows at least some of the particles incorporated into the material to be bared, without this embrittling the structure of the final material.

Whatever the method of obtaining them, the materials of the invention may advantageously be used as a heterogeneous catalyst, and especially in the field of the refining of petroleum cuts, the catalytic denitrification of combustion gas, particularly in the automobile field, or else in the catalysis of oxidation or transesterification reactions, in particular in the catalytic oxidation of volatile organic compounds or in the basic catalysis of transesterification of carbonates. The materials of the invention may also be used as fillers for reinforcing polymer matrices or films.

The various advantages of the present invention will become more explicitly apparent in the light of the illustrative but nonlimiting examples present below.

### EXAMPLE 1

### Preparation of an Ordered Mesoporous Material Incorporating Particles Based on Yttrium Oxide and Palladium in the Oxidation State 0

A—Preparation of a Colloidal Dispersion of YPd(OH)₃

### Particles

Placed in a beaker, with stirring at 20° C., were 65.35 g of an yttrium nitrate solution characterized by an La₂O₃ equivalent concentration of 21.78 wt % (this La₂O₃ equivalent concentration corresponds to an m/M mass ratio where M represents the mass of La₂O₃ obtained by calcining a mass M of the solution in air), representing 0.126 mol of yttrium.

Next, 21.8 g (i.e. 0.105 mol) of citric acid (M=192 g/mol) were added to this solution, and then demineralized water was added until obtaining a volume of 250 cm³ of an aqueous mixture characterized by a citric acid/yttrium molar ratio of 0.83.

165 cm³ of an aqueous ammonia solution containing 3.21 mol per liter were added to this mixture, with stirring, at a rate of 2.5 cm³ per minute using a metering pump. A visually clear mixture of 8.25 pH was then obtained.

This mixture was left, with stirring, at 20° C. for 60 min.

Following this stirring, a 50 ml aliquot of the dispersion thus prepared was removed. To this aliquot was added 0.3 ml of a solution of Pd(NO₃)₂ containing 0.98 mol per liter of palladium, sold by Comptoir Lyon Allemand Louyot.

Following this addition, the pH was lowered and readjusted by adding a 3.21M NH₄OH solution so as to obtain a value 8.25. This operation was repeated five times with a final addition of 0.03 ml of Pd(NO₃)₂, representing an addition of a total volume of 1.53 ml of the Pd(NO₃)₂ solution. The total amount of 3.21M aqueous ammonia added was 3.2 ml. The pH of the dispersion in fine obtained was measured to be 8.05.

The dispersion obtained after these various treatments was fractionated and immediately transferred into closed vessels (Parr bombs) heated at 120° C. for 16 hours.

After this heat treatment, a colloidal dispersion was obtained in each Parr bomb. These dispersions were joined together.

After cooling down to room temperature, the dispersion obtained was washed by ultrafiltration. To do this, distilled water was added to the dispersion in an amount of twice its volume, then the dispersion was ultrafiltered over a 3 kD membrane until reobtaining the initial volume of the dispersion, and this operation was repeated once. The dispersion was then concentrated by ultrafiltration.

The dispersion obtained was metered by the evaporation and calcining at 1000° C. of a given volume of dispersion. An equivalent concentration of 0.82 mol per liter of yttrium was determined.

Using transmission electron cryomicroscopy, colloids having a mean size of around 3 nm were observed in the dispersion obtained, these colloids being separate and having a monodisperse size distribution.

B—Production of the Pd:Y₂O₃:SiO₂Mesoporous Material

A solution (A) was obtained by obtained by adding, in a beaker, with stirring at 20° C., the following:

1 g of Brij 56 (grafted polyethylene oxide sold by Fluka);

5.5 g of water; and

8.55 g of a 0.5M HCl aqueous solution.

This solution, left with stirring, was transferred to a sealed vessel heated to 35° C.

An aqueous solution (B) of 15 ml of sodium silicate containing 0.4M of SiO₂ and an “SiO₂/Na₂O” molar ratio of 3 was also produced. The solution B was heated to 35° C.

Next, the solution (B) was added in one go to the solution (A), so as to obtain a visually transparent mixture of pH 2.

Next, added in one go to this mixture were 7.19 ml of the colloidal dispersion prepared beforehand, representing 5.9 mmol of yttrium added. The pH of the mixture obtained was measured to be 6.3.

This mixture, initially entirely clear, became cloudy over the course of time with the formation of a precipitate. The stirring was left to continue for 16 hours at 35° C.

Next, the mixture was heated at 80° C. for 16 hours in a sealed vessel so as to carry out maturing at temperature.

After this maturing step, the mixture was centrifuged at 4500 rpm for 15 minutes. The centrifugation pellet obtained was redispersed in 40 ml of a solution of demineralized water and the dispersion obtained was again centrifuged at 4500 rpm for 15 minutes.

Following this second centrifuging step, the centrifugation pellet obtained was dried at 60° C. for 16 hours, and then left to cool.

The solid obtained was calcined in nitrogen with a temperature rise of 1° C. per minute from 20° C. to 500° C. and the temperature of 500° C. was maintained at 500° C. for 6 hours.

The solid obtained was left to cool and then calcined, this time in air, with the same temperature profile.

The product obtained showed a hexagonal mesostructure in transmission electron microscopy.

### EXAMPLE 2

### Preparation of an Ordered Mesoporous Material Incorporating Particles Based on a Mixed LaFeO₃ Oxide of Perovskite Structure

A—Preparation of a Colloidal Dispersion of Mixed Lanthanum-iron Hydroxide Particles

Placed in a beaker, with stirring at 20° C., were 101 g (i.e. 0.25 mol) of Fe(NO₃)₃.9H₂O and 105.94 g of an aqueous solution of lanthanum nitrate La(NO₃)₃ characterized by a concentration of 2.36 mol per liter (which represents 0.25 mol of lanthanum nitrate added). Demineralized water was added until a volume of 715 ml was obtained.

50 ml of the solution thus obtained were then added, with stirring, to 6.1 g of citric acid (C₆H₈O₇.H₂O) so as to obtain a mixture characterized by a citric acid/metal molar ratio of 0.83/1. 10M aqueous ammonia was then added, with stirring, to this mixture so as to obtain a pH of 6.3.

The dispersion obtained was fractionated and immediately transferred into sealed containers (Parr bombs) heated at 120° C. for 16 hours.

After this heat treatment, a colloidal dispersion was obtained in each Parr bomb. These dispersions were joined together.

After cooling down to room temperature, the dispersion obtained was washed by ultrafiltration. To do this, distilled water was added to the dispersion in an amount of twice its volume, then the dispersion was ultrafiltered over a 3 kD membrane until the initial volume of the dispersion was obtained again, and this operation was repeated once. Next, the dispersion was concentrated by ultrafiltration until a lanthanum-iron concentration of 0.7 mol per liter was obtained.

Using transmission electron cryomicroscopy, colloids were observed in the dispersion obtained, these having a mean size of around 3 nm and being completely separate.

After ultracentrifugation at 50 000 rpm for 6 hours of an aliquot of the dispersion, an ultracentrifugation pellet was collected. The chemical analysis of this pellet revealed an La/Fe molar ratio of 1 within the particles.

B—Production of the La₂O₃:SiO₂ Mesoporous Material

A solution (A) was obtained by obtained by adding, in a beaker, with stirring at 20° C., the following:

1 g of Brij 56 (grafted polyethylene oxide sold by Fluka);

5.5 g of water; and

8.55 g of a 0.5M HCl aqueous solution.

This solution, left with stirring, was transferred to a sealed vessel heated to 35° C.

An aqueous solution (B) of 15 ml of sodium silicate containing 0.4M of SiO₂ and an “SiO₂/Na₂O” molar ratio of 3 was also produced. The solution B was heated to 35° C.

Next, the solution (B) was added in one go to the solution (A), so as to obtain a visually transparent mixture of pH 2.

Next, added in one go to this mixture were 7.19 ml of the colloidal dispersion prepared beforehand, representing 5.95 mmol of lanthanum and of iron added. The pH of the mixture obtained was measured to be 5.9.

This mixture, initially entirely clear, became cloudy over the course of time with the formation of a precipitate. The stirring was left to continue for 16 hours at 35° C.

Next, the mixture was heated at 80° C. for 16 hours in a sealed vessel so as to carry out maturing at temperature.

After this maturing step, the mixture was centrifuged at 4500 rpm for 15 minutes. The centrifugation pellet obtained was redispersed in 40 ml of a solution of demineralized water and the dispersion obtained was again centrifuged at 4500 rpm for 15 minutes.

Following this second centrifuging step, the centrifugation pellet obtained was dried at 60° C. for 16 hours, and then left to cool.

The solid obtained was calcined in nitrogen with a temperature rise of 1° C. per minute from 20° C. to 500° C. and the temperature of 500° C. was maintained at 500° C. for 6 hours.

The solid obtained was left to cool and then calcined, this time in air, with the same temperature profile.

The product obtained showed a hexagonal mesostructure in transmission electron microscopy.

1. A thermally stable mesostructured material, the walls of the mesostructure of which comprise:
(a) a mineral matrix; and
(b) dispersed within this mineral matrix (a), nanoscale particles based on at least one rare earth E and on at least one transition element M different from this rare earth, in which particles:
(b1) the rare earth E is at least partly in the form of an oxide, hydroxide and/or oxyhydroxide, the transition element M then being at least partly in the oxidation state 0; or
(b2) the rare earth E and the transition element M are at least partly present in the form of a hybrid oxide possessing a crystal structure.

2. The material as claimed in claim 1, characterized in that it has at least one organized structure chosen from:
mesoporous mesostructures of P63/mmc three-dimensional hexagonal symmetry, two-dimensional hexagonal symmetry or Ia3d, Im3m or Pn3m three-dimensional cubic symmetry; or
mesostructures of the vesicular, lamellar or vernicular type.

3. The material as claimed in claim 1 or claim 2, characterized in that the overall wall thickness of the mesostructure is between 2 nm and 20 nm.

4. The material as claimed in any one of claims 1 to 3, characterized in that it is an ordered mesoporous material in which the mean pore diameter is between 2 nm and 30 nm.

5. The material as claimed in any one of claims 1 to 4, characterized in that it has a specific surface area of between 500 and 3000 m²/cm³.

6. The material as claimed in any one of claims 1 to 5, characterized in that the mineral matrix (a) is based on silica, zirconia, alumina and/or titania.

7. The material as claimed in claim 6, characterized in that the mineral matrix (a) consists predominantly of silica.

8. The material as claimed in any one of claims 1 to 7, characterized in that the particles (b) are particles of spherical or slightly anisotropic morphology, at least 50% of the population of which possesses a mean diameter of between 2 nm and 25 nm, the size distribution of said particles being monodisperse.

9. The material as claimed in any one of claims 1 to 8, characterized in that at least some of the nanoscale particles dispersed within the binding mineral phase are in contact with the porous parts constituting the internal space of the material.

10. The material as claimed in any one of claims 1 to 9, characterized in that the particles (b)/mineral phase (a) volume ratio is between 5% and 95%.

11. The material as claimed in any one of claims 1 to 10, characterized in that the particles (b) comprise:
an oxide, hydroxide and/or oxyhydroxide of the rare earth E; and
the transition element M at least partly in the oxidation state 0.

12. The material as claimed in claim 11, characterized in that at least some of the transition element M present in the oxidation state 0 is localized on the periphery of the particles (b).

13. The material as claimed in claim 11 or claim 12, characterized in that the rare earth E is chosen from yttrium, lanthanum, cerium, praseodymium, neodymium, samarium, europium, gadolinium, terbium and dysprosium.

14. The material as claimed in any one of claims 11 to 13, characterized in that the transition element M present in the oxidation state 0 is chosen from rhodium, platinum, palladium, ruthenium, cobalt, copper, silver, nickel and manganese.

15. The material as claimed in any one of claims 11 to 14, characterized in that the metal M/rare earth E molar ratio is between 0.002 and 0.2.

16. The material as claimed in any one of claims 1 to 10, characterized in that the particles (b) are based on a mixed oxide possessing a crystal structure incorporating cations of the rare earth E and cations of said transition element M.

17. The material as claimed in claim 16, characterized in that the mixed oxide has a structure of the perovskite or pyrochlore type, or a structure similar to that of K₂NiF₄.

18. The material as claimed in claim 16 or claim 17, characterized in that the rare earth E is chosen from yttrium, lanthanum, cerium, praseodymium, neodymium, samarium, europium, gadolinium, terbium and dysprosium.

19. The material as claimed in any one of claims 16 to 18, characterized in that the metal M is chosen from titanium, chromium, manganese, iron, cobalt, nickel, copper, zinc, zirconium, silver or a rare earth different from the rare earth E.

20. The material as claimed in any one of claims 16 to 19, characterized in that the particles (b) based on a mixed oxide furthermore incorporate a transition element M′ present in the oxidation state 0.

21. The material as claimed in claim 20, characterized in that at least part of the transition element M′ present in the oxidation state 0 is localized on the periphery of the particles (b).

22. The material as claimed in claim 20 or claim 21, characterized in that the transition element M′ is chosen from rhodium, platinum, palladium or ruthenium, silver, cobalt, copper or nickel.

23. A process for preparing a material as claimed in any one of claims 1 to 22, characterized in that it comprises the steps consisting in:
(1) bringing together, in a medium containing a templating agent:
(i) nanoscale particles comprising at least one oxide, hydroxide or oxyhydroxide of the rare earth E, said particles being complexed by at least one salt of the metal M; or nanoscale particles comprising at least one mixed oxide or mixed hydroxide of the rare earth E and of the metal M; and
(ii) a mineral precursor, capable of leading, in said medium, to the formation of an insoluble mineral phase under suitable pH conditions;
(2) leaving said mineral phase to form from said precursor (ii), or if necessary to make said mineral phase form by adjusting the pH, whereby a mesostructure whose walls are formed from said mineral phase trapping at least some of the initially introduced nanoscale particles is obtained;
(3) if necessary, subjecting the mesostructure obtained from step (2) to a heat treatment step and/or to a reduction step, this or these steps being carried out, as the case may be, so that, after this or these treatments, the particles present within the walls of the mesostructure comprise particles meeting the definition of the particles (b) of claim 1; and
(4) if necessary, removing the templating agent optionally present in the pores of the mesostructure obtained from these various steps.

24. The process for preparing a material as claimed in any one of claims 11 to 15, characterized in that it comprises the steps consisting in:
(1) bringing together, in a medium containing a templating agent:
(i) nanoscale particles comprising at least one oxide, hydroxide or oxyhydroxide of the rare earth E, said particles being complexed by at least one salt of the metal M; or nanoscale particles comprising one mixed oxide and/or mixed hydroxide of the rare earth E and of the transition element M; and
(ii) a mineral precursor, capable of leading, in said medium, to the formation of an insoluble mineral phase under suitable pH conditions;
(2) leaving said mineral phase to form from said precursor (ii), or if necessary to make said mineral phase form by adjusting the pH, whereby a mesostructure whose walls are formed from said mineral phase trapping at least some of the initially introduced nanoscale particles is obtained;
(3) subjecting the mesostructure obtained after step (2) to a heat treatment/reduction step carried out so as to form the metal M in the oxidation state 0 from at least some of the salt of the metal M; and
(4) if necessary, removing the templating agent optionally present in the pores of the mesostructure obtained from these various steps.

25. The process for preparing a material as claimed in any one of claims 16 to 19, characterized in that it comprises the steps consisting in:
(1) bringing together, in a medium containing a templating agent:
(i) nanoscale particles comprising at least one mixed oxide or mixed hydroxide of the rare earth E and of the metal M; and
(ii) a mineral precursor, capable of leading, in said medium, to the formation of an insoluble mineral phase under suitable pH conditions;
(2) leaving said mineral phase to form from said precursor (ii), or if necessary to make said mineral phase form by adjusting the pH, whereby a mesostructure whose walls are formed from said mineral phase trapping at least some of the initially introduced nanoscale particles is obtained;
(3) if necessary, subjecting the mesostructure obtained from step (2) to a heat treatment step carried out, where appropriate, so that, after this heat treatment, the particles present within the walls of the mesostructure comprise a mixed oxide of crystal structure meeting the definition (b1) of claim 1; and
(4) if necessary, removing the templating agent optionally present in the pores of the mesostructure obtained from these various steps.

26. The process for preparing a material as claimed in any one of claims 20 to 22, characterized in that it comprises the steps consisting in:
(1) bringing together, in a medium containing a templating agent:
(i) nanoscale particles comprising at least one mixed oxide or mixed hydroxide of the rare earth E and of the metal M, said particles being complexed by at least one salt of the metal M′; and
(ii) a mineral precursor, capable of leading, in said medium, to the formation of an insoluble mineral phase under suitable pH conditions;
(2) leaving said mineral phase to form from said precursor (ii), or if necessary to make said mineral phase form by adjusting the pH, whereby a mesostructure whose walls are formed from said mineral phase trapping at least some of the initially introduced nanoscale particles is obtained;
(3) subjecting the mesostructure obtained from step (2) to a heat treatment/reduction step carried out so that, after this treatment, the particles present within the walls of the mesostructure comprise a mixed oxide of crystal structure meeting the definition (b1) of claim 1, and at least some of the metal M′ in the oxidation state 0; and
(4) if necessary, removing the templating agent optionally present in the pores of the mesostructure obtained from these various steps.

27. The process as claimed in any one of claims 23 to 26, characterized in that the medium employed is an aqueous medium.

28. The process as claimed in any one of claims 23 to 27, characterized in that the templating agent used is a nonionic amphiphilic agent of the block copolymer type, chosen from grafted poly(ethylene oxide)/poly(propylene oxide)/poly(ethylene oxide) triblock copolymers or polyethylene oxides.

29. The process as claimed in any one of claims 23 to 28, characterized in that the (templating agent)/(metal cations present in the particles (i)+mineral precursor (ii)) molar ratio is generally between 0.05 and 3.

30. The process as claimed in any one of claims 23 to 29, characterized in that the particles (i) are introduced in the form of a colloidal dispersion within which the particles possess, for at least 50% of their population, a mean diameter of between 1 nm and 25 nm.

31. The process as claimed in any one of claims 23 to 30, characterized in that the mineral precursor (ii) is an alkali metal silicate, preferably sodium silicate.

32. The process as claimed in any one of claims 23 to 31, characterized in that the templating procedure is carried out at a temperature of between 15° C. and 90° C.

33. The process as claimed in any one of claims 23 to 32, characterized in that the step for forming the mineral phase from the mineral precursor comprises a maturing step carried out at a temperature of between 15° C. and 85° C.

34. The process as claimed in any one of claims 23 to 33, characterized in that the mesostructured solid obtained after steps (1), (2) and optionally (3) and/or (4) is subjected to a subsequent heat treatment, especially to a calcining treatment.

35. The process as claimed in either of claims 23 and 24, characterized in that, subsequent to the templating agent elimination, a partial chemical etching of the mineral phase is carried out.

36. The use of a material as claimed in any one of claims 1 to 22, or of a material that can be obtained by the process as claimed in any one of claims 23 to 35, as heterogeneous catalyst for the denitrification of combustion gas, as catalyst for refining a petroleum cut, as catalyst for an oxidation reaction, as a transesterification catalyst or as a filler for reinforcing a polymer matrix or a film.
